(19) United States
(12) Patent Application Publication
(10) Pub. No.: US 2007/0147577 A1
(43) Pub. Date: Jun. 28, 2007

(54) RADIATION TOMOGRAPHIC IMAGING APPARATUS AND RADIATION TOMOGRAPHIC IMAGING METHOD

(76) Inventor: Masaru Seto, Tokyo (JP)

Correspondence Address: Patrick W. Rasche;Armstrong Teasdale LLP, Suite 2600, One Metropolitan Square, St. Louis, MO, 63102, US

(21) Appl. No.: 11/613,743

(22) Filed: Dec. 20, 2006

(30) Foreign Application Priority Data
Dec. 28, 2005 (JP) 2005-376790

(51) Int. Cl. A61B 6/00; G01N 23/00; G21K 1/12; H05G 1/60

(52) U.S. Cl. 378/8

(57) ABSTRACT

There is provided a radiation tomographic imaging apparatus and a radiation tomographic imaging method which allows the ECG information and the X-ray tube current value to be monitored while obtaining required projection data for the image reconstruction. The radiation tomographic imaging method for forming a tomographic image of a subject by means of the radiation from the radiation source comprises the ECG wave output step for measuring the heartbeat of the heart of the subject to output as the ECG wave signal; a variable output step for varying the radiation output based on the ECG wave signal; a determining step for determining the tomographic image reconstructed based on the projection data obtained by the radiation output having been varied is good or no good; and a displaying step for displaying the ECG wave signal, the radiation output, and the reconstruction data area for forming a tomographic image if the image is no good.

### CROSS REFERENCE TO RELATED APPLICATIONS

This application claims the benefit of Japanese Application No. 2005-376790 filed Dec. 28, 2005.

### BACKGROUND OF THE INVENTION

The present invention relates to a radiation tomographic imaging method for performing a tomographic imaging of a subject by emitting radiation from the circumference of the subject, namely the patient to obtain data and to process the data. More specifically the present invention relates to the reconstruction of the image of a heart area.

As the diagnostic apparatus for a lesion in a subject, a radiation tomographic imaging apparatus, such as an X-ray CT apparatus, for obtaining a tomographic image of a subject is widely used for the diagnosis. The X-ray CT apparatus is also widely used in the imaging of the heart area.

When imaging the heart area, because the heart is beating all the time, the patient has an electrocardiogram attached thereon to monitor the function state of the heart such as the systolic and diastolic period, while emitting the radiation. The reconstruction method of the image of the heart is referred to as ECG (electrocardiogram) reconstruction method, which includes the prospective ECG method (prospective EGA) and the retrospective ECG method (retrospective ECG). The retrospective ECG method is used in JP-A-2004-173923.

In the prospective ECG method, the image is reconstructed from the projection data obtained from the ECG information at a constant interval, in order to view the image of heartbeat phase set prior to projection of radiation. In this method the electric current flowing through the radiation tube is altered so as to improve the S/N ratio at the timing of the phase of the end of diastole or the end of systole so that the radiation exposure to the subject altogether is attempted to be decreased. However, when the heartbeat is tachycardiac if subject is tense by holding the breath or when in arrhythmia, desired heartbeat phase may or may not be well imaged or there may be some motion artifacts.

In the retrospective ECG method, the electrocardiographic information is acquired while at the same time radiation is emitted to obtain the projection data. After the projection of radiation, required projection data of the heartbeat phase from the electrocardiographic information is retrieved in order to perform the image reconstruction. For the heartbeat phase to be extracted the minimal heartbeat phase may be selected, or the projection data is extracted at the phase required for the diagnosis. This allows imaging the heart area with the minimal motion artifacts caused by the body move. However, in the retrospective ECG method, the radiation exposure dose to the subject is higher because of sequential projection of X-ray.

There is a need of an apparatus or a method for specifying the projection data required for the image reconstruction by taking into account the ECG information and tube current value, when obtaining the projection data by changing the tube current in correspondence with the phase of predetermined heartbeat.

### SUMMARY OF THE INVENTION

The subject of the present invention therefore is to provide a radiation tomographic imaging apparatus and radiation tomographic imaging method which allows the operator to confirm the ECG information as well as the radiation tube current value without any complex operation, and to specify the required projection data for the image reconstruction.

The radiation tomographic imaging apparatus in accordance with a first aspect includes an electrocardiogram for measuring the heartbeat of the heart of a subject and for outputting electrocardiographic wave signals, an input unit for receiving a predetermined phase of the heartbeat, a variable output unit for varying the output of radiation based on the ECG wave signal and predetermined phase, and a display unit for displaying the ECG wave signal, the radiation output, and the reconstruction data area for forming a tomographic image. With this arrangement the operator (a physician or a radiologist) is allowed to confirm the reconstruction data area for forming a tomographic image while taking into account the ECG wave signal and the radiation output.

In the radiation tomographic imaging apparatus in accordance with a second aspect, the display unit displays graphically the ECG wave signal, the radiation output state, and the reconstruction data area. With this arrangement the operator is allowed to intuitively confirm the ECG wave signal, the radiation output state, and the reconstruction data area.

The radiation tomographic imaging apparatus in accordance with a third aspect further includes a changing means for changing the area of the reconstruction data. With this arrangement the operator is allowed to change the area for the reconstruction data for forming the tomographic image while taking into account the ECG wave signal and the radiation output.

In the radiation tomographic imaging apparatus in accordance with a fourth aspect, a changing means manipulates the area of the reconstruction data displayed on the display unit in the direction of time axis. With this arrangement the operator is allowed to replace the reconstruction data area which is not desirable due to for example arrhythmia with another reconstruction data area, and the area with another so as to avoid an inappropriate state of the radiation output.

The radiation tomographic imaging apparatus in accordance with a fifth aspect further includes a plurality of areas of reconstruction data, in which an appropriate number of areas can be changed by the changing means in the direction of time axis at the same time. With this arrangement a tomographic image of the heart of the required phase can be obtained.

The radiation tomographic imaging apparatus in accordance with a sixth aspect further includes a plurality of areas of reconstruction data, in which the changing means deletes or adds at least one area of reconstruction data to change the reconstruction data area. A clear tomographic image can be obtained by deleting an area which becomes an obstacle when reconstructing a tomographic image.

The radiation tomographic imaging apparatus in accordance with a seventh aspect further includes an input unit for inputting a predetermined phase among heartbeats, wherein the output of the radiation from the variable output unit varies at the predetermined phase from the ECG wave signal. With this arrangement the dose of radiation exposure to the subject can be minimized while the operator can obtain the projection data with the required heart phase.

In the radiation tomographic imaging apparatus in accordance with an eighth aspect, the radiation includes X-ray. Because of very high signal-to-noise ratio (SNR), a minute difference of X-ray permeability can be detected on the image.

The radiation tomographic imaging method in accordance with a ninth aspect, includes an electrocardiographic wave output step for measuring the heartbeat of the heart of the subject to output as ECG wave signal, a phase input step for inputting a predetermined phase of the heartbeats of the heart, a variable output step for varying the output of radiation based on the ECG wave signal and the predetermined phase, a determination step for determining whether good or no good is the tomographic image reconstructed based on the projection data obtained from the varied output of the radiation, and a display step for displaying the ECG wave signal, the radiation output, and the reconstruction data area for forming a tomographic image at the same time when the image is no good. With this arrangement the operator is allowed to confirm the area of the reconstruction data for forming a tomographic image while taking into consideration the ECG wave signal and the radiation output.

In the radiation tomographic imaging method in accordance with a tenth aspect, the display step graphically displays the ECG wave signal, the output state of the radiation, and the area of the reconstruction data. With this arrangement the operator is allowed to intuitively confirm the ECG wave signal, the output state of the radiation, and the area of the reconstruction data.

The radiation tomographic imaging method in accordance with an eleventh aspect further includes a changing step for changing the area of the reconstruction data. With this arrangement the operator is allowed to change the area of the reconstruction data for forming a tomographic image while taking into consideration the ECG wave signal and the output of the radiation.

In the radiation tomographic imaging method in accordance with a twelfth aspect, the changing step changes the area of the reconstruction data by operating the area of the reconstruction data displayed on the display unit in the direction of time axis. With this arrangement the operator is allowed to replace the reconstruction data area which is not desirable due to for example arrhythmia with another reconstruction data area, and the area with another so as to avoid an inappropriate state of the radiation output.

The radiation tomographic imaging method in accordance with a thirteenth aspect further includes a plurality of areas of the reconstruction data, and the changing means may change an arbitrary number of areas in the direction of the time axis at the same time. With this arrangement a tomographic image at the required phase of the heart can be easily obtained.

The radiation tomographic imaging method in accordance with a fourteenth aspect further includes a plurality of areas of the reconstruction data, in which the changing means deletes or adds at least one area of reconstruction data to change the reconstruction data area. With this arrangement a tomographic image at the required phase of the heart can be easily obtained.

The radiation tomographic imaging method in accordance with a fifteenth aspect further includes a reconstruction step for reconstructing a tomographic image by using the reconstruction data of the area changed in the changing step, among the projection data obtained by the output of the radiation varied. With this arrangement the operator is allowed to confirm the tomographic image with the reconstruction data of the changed area.

The radiation tomographic imaging method in accordance with a sixteenth aspect further includes a phase input step for inputting a predetermined phase of the heartbeat of the hear, so that the output of the radiation by the variable output step varies in the predetermined phase from the ECG wave signal. With this arrangement the exposure dose of the radiation to the subject for example can be minimized while at the same time the operator may obtain the projection data of the required state of the heart.

The radiation tomographic imaging method in accordance with a seventeenth aspect may obtain a tomographic image of the heart by displacing the source of radiation and the moving table carrying thereon the subject in synchronization, based on the ECG wave signal, namely a helical scan.

In the radiation tomographic imaging method in accordance with an eighteenth aspect the radiation includes X-ray. Because of very high signal-to-noise ratio (SNR), a minute difference of X-ray permeability can be detected on the image.

The radiation tomographic imaging apparatus or method in accordance with the present invention allows the increase of efficiency of the diagnosis of a subject because of the improved operability at the time when confirming a tomographic image of the heart by the operator. The apparatus or method may obtain a tomographic image with the minimal motion artifact due to the body move. In addition, the dose of radiation exposure to the subject can be decreased.

### BRIEF DESCRIPTION OF THE DRAWINGS

FIG. 1 is an overview of the X-ray CT apparatus 1 in accordance with the preferred embodiment of the present invention;

FIG. 2 is a flow chart illustrating the contents of electrocardiograph synchronization scan processing in accordance with the preferred embodiment of the present invention;

FIG. 3 is a schematic diagram illustrating the range of projection data when performing a helical scan; and

FIG. 4 is a graphic display screen displayed on a monitor 56.

### DETAILED DESCRIPTION OF THE INVENTION

[Overview of an X-Ray CT Apparatus]

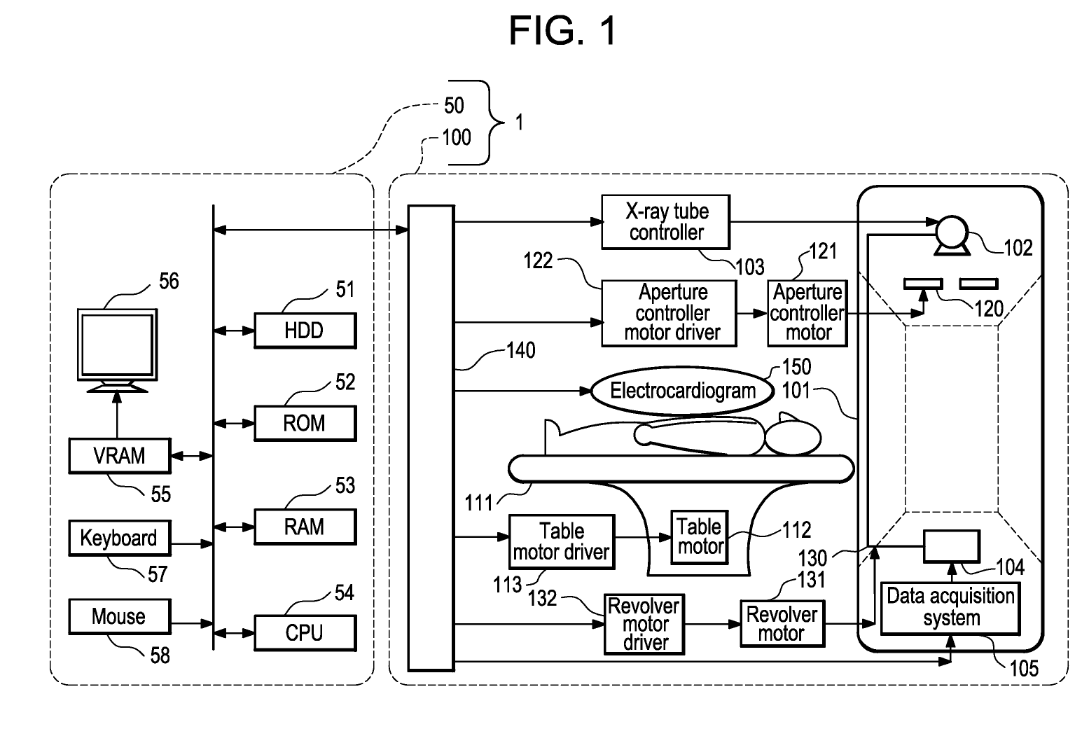

Now referring to FIG. 1, there is shown an overview of an X-ray CT apparatus 1 in accordance with the preferred embodiment. As shown in the figure, the apparatus includes a gantry 100 for emitting X-ray to the subject and for detecting X-ray transmitted through the subject, and an operation console 50 for reconstructing an X-ray tomographic image based on the data transferred from the gantry 100 and for outputting and displaying.

The gantry 100 includes a CT controller unit 140 for managing the entity and is connected to a variety of equipment as will be described below.

Inside the gantry 100, there are provided an X-ray tube 102 which is the source of X-ray, an X-ray tube controller 103 connected to the X-ray tube 102, a collimator 120 having an aperture for limiting the radiation range of the X-ray, an aperture controller motor 121 for adjusting the aperture width of the collimator 120, and an aperture controller motor driver 122 for driving the aperture controller motor 121. The X-ray passing through the collimator 120 is shaped to be an X-ray beam in a form of fan (fan-beam) along with the revolving direction of the gantry 100, made by the limitation of the X-ray radiation range by the collimator 120. The subject (patient) lying on a table 111 is driven in the direction of body axis of the subject (patient) (or in the direction of z-axis, which in general coincides with the direction of the body axis of the subject) by a table motor 112. The table motor 112 is driven by a table motor driver 113.

Also inside the gantry 100, there is provided an X-ray detector unit 104, which has detector channels comprised of a plurality of rows in the direction of elements (identical to the direction of z-axis) of a plurality of detectors, spanning to a length relying on the fan angle (approximately 60 degrees in usual configuration). The X-ray detector unit 104 may be formed by a combination of scintillators and photodiodes, for example. The configuration is not limited thereto, for example the X-ray detector unit 104 may be comprised of semiconductor X-ray detector elements using Cadmium-Tellurium (CdTe), or may be comprised of X-ray detector elements of the type ionization chamber using Xenon (Xe) gas.

The gantry 100 includes a plurality of data acquisition systems (DAS) 105 each acquiring the output from the detector channel as projection data. The data acquisition system 105 is comprised of one single or a plurality of units (for example, 4, 8, 16, or 32 units), each connected to the X-ray detector unit 104. For example, a unit called as 4 DAS, which has four data acquisition systems, in general may have four rows of detector channels placed in the direction of elements, and is able to acquire four images of slice during the time the X-ray tube 102 revolves one turn. The X-ray tube 102 and the X-ray detector unit 104 are placed each other in the opposite side of the bore or the subject. A revolving unit 130 is provided so as to revolve around the subject with the opposing geometric relationship of the X-ray tube 102 and the X-ray detector unit 104 being kept. The revolving unit 130 is connected to a revolver motor 131 and a revolver motor driver 132 and is controlled to rotate one turn per 0.3 second to 1.0 second by the revolver motor driver 132. It should be noted here that there is a gantry 100 having X-ray detector units 104 placed on the entire circumference of the gantry with an X-ray tube 102 solely turning round. The present invention can be applied to the system having only the X-ray tube 102 revolving.

In addition in the preferred embodiment, an electrocardiogram 150 which converts the heartbeat movement into electric signal is attached to the subject for confirming the heartbeat rate of the subject. This is used for the electrocardiographic synchronization scan as will be described later.

The CT controller unit 140 is connected to the operation console 50 so as to communicate each other. Upon instruction of the operation console 50, the CT controller unit 140 outputs control signals to the X-ray tube controller 103, table motor driver 113, aperture controller motor driver 122, revolver motor driver 132, data acquisition system 105. The data acquired by the data acquisition system 105 is transferred to the operation console 50 for the image reconstruction.

The X-ray CT apparatus 1 provides a full scan mode, in which an image is reconstructed from the projection data of 360 degrees, and a half scan mode, in which an image is reconstructed from the projection data of 180 degrees plus fan angle, such that the user may arbitrarily selected. In the full scan mode a high quality tomographic image can be reconstructed, while in the half scan mode the image quality of the tomographic image may be somewhat sacrificed but the scan speed is faster, and therefore the half scan mode has such advantage that the dose of X-ray exposure to the subject is decreased.

The operation console 50 is a so-called workstation, which, as shown in the figure, has a ROM 52 for storing the boot program, a RAM 53 that works as the main memory device, and a CPU 54 that controls the entire device.

A hard disk drive 51 stores therein an operating system, and an image processing program, which gives the gantry 100 a various instruction, reconstructs an X-ray tomographic image based on the data received from the gantry 100, and performs display. A VRAM 55 is a memory for expanding the image data to be displayed, and the image data expanded therein can be displayed on a monitor 56. The operation is through a keyboard 57 and a mouse 58.

In the X-ray CT apparatus 1 as have been described above, the acquisition of the projection data may be performed as follows.

First, the table motor 112 which mounts the subject thereon placed within the bore of the gantry revolving unit 130 translates in the direction of z-axis at a predetermined velocity. The revolving unit 130 revolves while the X-ray tube 102 emits X-ray beam onto the subject. The transmitted X-ray is detected by the X-ray detector unit 104. The detection of the transmission X-ray is performed by the X-ray tube 102 and the X-ray detector unit 104 turning around the subject (i.e., by varying the radiation angle (view angle)) in a plurality of view angles N (for example N=1,000), for 180 degrees plus fan angle. The transmission X-ray thus detected is converted to a digital value by the data acquisition system 105 to transfer to the operation console 50 as projection data. This sequence makes a unit and is referred to as ‘one scan’. As can be seen, the projection data acquisition by moving the table 111 at a predetermined velocity in synchronism with the change of the radiation angle to move the scanning position (the X-ray tube 102 and the X-ray detector unit 104 revolves around the subject in a helicoidal trajectory) is referred to as a ‘helical scan’ method. In the preferred embodiment, although the helical scan is described, the same is applied to the axial scan method, in which the table motor 112 is stepped sequentially in the direction of z-axis and the gantry revolving unit 130 is rotated around the subject to acquire the projection data.

The operation console 50 displays on the monitor 56 the input information, the required procedure for the image reconstruction, or a tomographic image reconstructed in accordance with the predetermined computation based on the Radon's principle for the transferred projection data.

[Electrocardiogram synchronization scan by the X-ray CT apparatus 1]

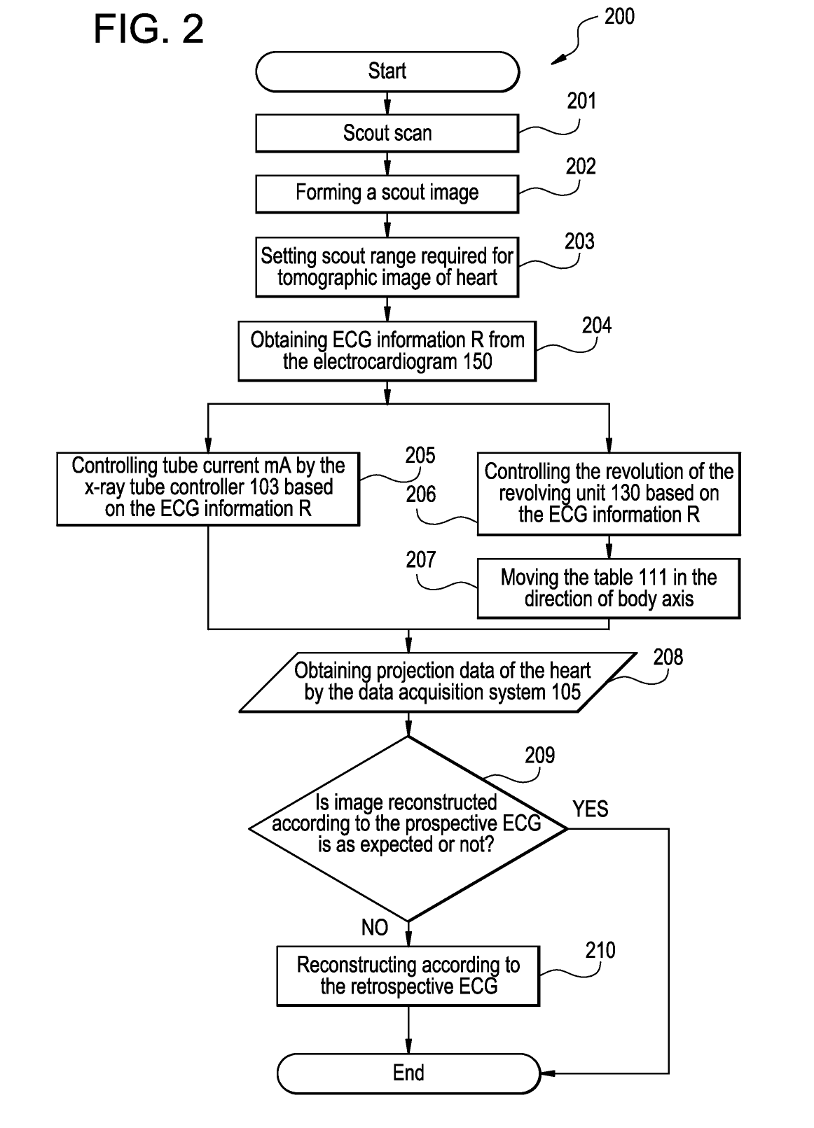

Now referring to the flow chart shown in FIG. 2, the electrocardiogram synchronization scan method 200 of the heart will be described in greater details.

A program implementing the flow chart of FIG. 2 is included in the image processing program stored in the hard disk drive 51 of the operation console 50, which is executed by the CPU 54 when loaded into the RAM 53.

The process example shown describes a scanning project for performing a helical scan on and around the heart for the purpose of the diagnosis of the heart. It is equally possible to conduct a scanning project for the purpose of the diagnosis of another organ at the same time, however for the sake of clarity, the diagnosis of the heart only will be described.

In step S201 the operator (a physician or a radiologist) uses the keyboard 57 and the mouse 58 for the input of predetermined information and confirms the input information on the monitor 56, then initiates a scout scan. A scout scan is a scan in that the X-ray tube 102 is held stationarily at a given position (i.e., the revolving unit 130 does not rotate so as to hold at a constant radiation angle) while the table 111 is displaced at a constant speed in the direction of the body axis, and the X-ray is continuously emitted to obtain the projection data (transmission data) to obtain one transmission image of the subject. The transmission image of the subject thus obtained is referred to as a scout image.

Upon reception of the execution instruction of a scout scan from the operation console 50, the gantry 100 performs the scout scan requested by the execution instruction. The operation console 50 receives the transmission image data transferred from the X-ray detector unit 104 and the data acquisition system 105 and stores the data in the RAM 53.

In step S202, the scout image stored in the RAM 53 is displayed on the monitor 56. The operator confirms the scout image displayed on the monitor 56 while at the same time setting the ECG synchronization scan start position and ECG synchronization scan end position by using the mouse 58 as preparation of the ECG synchronization scan of the heart (step S203). The segment between the start position and end position of the ECG synchronization scan is the ECG synchronization scan segment. Then the operator instructs the execution of an ECG synchronization scan using the keyboard 57 and the mouse 58.

In step S204, The ECG information R is detected from the electrocardiogram 150. The moving state of the heart (systolic phase, diastolic phase) can be recognized from the ECG information R. The interval between a preceding heartbeat (peak R of QRS wave) and a succeeding peak (peak R of QRS wave) of the ECG information R is usually referred as to RR interval. The operator sets the phase as the relative position to the RR interval (percent setting), and the projection data will be extracted about the phase set. For example, if you want to confirm the tomographic image at the end of a diastolic phase of the heart, the phase is to be set to 70 to 80 percent using the keyboard 57. If you want to confirm the tomographic image at the end of a systolic phase of the heart, the phase is to be set to 35 to 45 percent using the keyboard 57. The setting value is sent through the CT controller unit 140 to the X-ray tube controller 103.

The operator also sets the tube current mA in addition to setting of the phase of the relative position to the RR interval. For example, when the output power of the X-ray tube 102 is 40 kW, the operator will set the X-ray power at MIN value (for example, approximately 0 kW to 10 kW) and the MAX value (for example, 20 kW to 30 kW). These MIN value and MAX value will also be sent through the CT controller unit 140 to the X-ray tube controller 103. In the MIN setting, it will be preferable to set at least some tube current mA. By doing this the projection data can be acquired for the image reconstruction even in case of arrhythmia.

In the following step S205, the X-ray tube controller 103 is controlled in correspondence with the ECG information R. More specifically, the X-ray tube controller 103 includes a high frequency inverter device, and the tube current mA flowing through the high frequency inverter device is controlled in synchronism with the heart beat cycle of the subject detected by the electrocardiogram 150 to vary the radiation intensity of X-ray from the X-ray tube 102. When the heart is expanding or shrinking, the projection data obtained by the data acquisition system 105 may have a larger motion artifact so that the data will be often inappropriate for the image reconstruction. In step S204, when the phase of the relative position to the RR interval is set to 75 percent, the X-ray tube controller 103 controls the X-ray tube 102 so that the X-ray output becomes MAX setting in the range of phase from 60 percent to 90 percent, and the X-ray tube controller 103 controls the X-ray tube 102 so that the X-ray output becomes MIN setting outside the range. It is preferable that the raising interval from the MIN to MAX setting of the X-ray output and the falling interval from the MAX to MIN setting of the X-ray output are as short as possible.

In step S206, in parallel to step S205, the revolving speed of the gantry revolving unit 130 is set to be in synchronism with the heart rate of the subject detected by the electrocardiogram 150. The value of the revolving speed displayed can be modified by the operator. Instead of directly using the detection output of the electrocardiogram 150 for the computation of the revolving speed of the gantry revolving unit 130, the operator inputs the heart rate via the keyboard 57, and the revolving speed of the revolving unit 130 can be calculated from the information input.

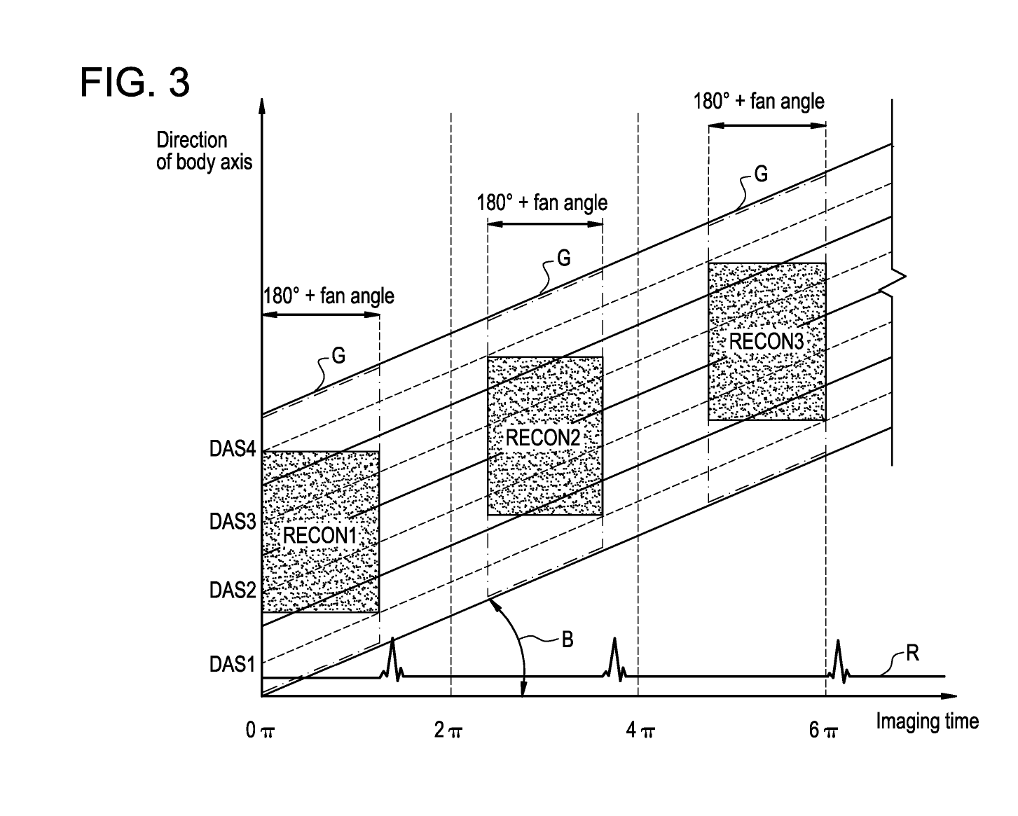

In step S207, the moving speed of the table 111 is controlled by the table motor 112 and the table motor driver 113 in correspondence with the revolving speed of the gantry revolving unit 130 determined in step S205. The moving speed of the table 111 will be determined, not only by the revolving speed of the gantry revolving unit 130, but also by the number of data acquisition system 105 (4 DAS, 8 DAS, etc.), and by the helical pitch for obtaining the tomographic image appropriate for the diagnosis of the heart. The term helical pitch herein refers to the amount of displacement of the table 111 when the gantry rotates by the acquisition angle of the projection data required for the image reconstruction of one tomographic image, obtained by one data acquisition system 105. Setting of helical pitch will be described with reference to FIG. 3.

In step S208, the projection data of the heart is acquired by the data acquisition system 105. The process steps from S205 to S208 are the steps of prospective ECG method.

The process steps described above can be illustratively described as follows. The heartbeat wave of the subject is input using the electrocardiogram 150 to measure the heart rate. Then the revolving speed of the revolving unit 130 is set such that it rotates by 180 degrees plus fan angle for example in one heart cycle to execute a scan. In such a scan, the initial radiation angle of the revolving unit 130 advances by the fan angle for each half of a heart cycle. The amount of the X-ray emitted from the X-ray tube 102 will be intensified only during the period required. By doing this the projection data required for the image reconstruction of one tomographic image in a specific phase (for example, systolic phase of the heart) of the heart beat of the subject can be extracted (the projection data for 180 degrees plus fan angle in case of the half scan mode, the projection data for 360 degrees in case of the full scan mode). The image reconstruction will be conducted based on the projection data thus extracted. With the reconstruction process described above, in theory, a clear tomographic image can be obtained which does not have any artifact caused by the heart beat.

There are cases where the heart beat of the subject is not stable. For example the heartbeat gradually increases because the patient undergoing the CT examination is holding the breath, or the heartbeat is unstable because of arrhythmia. In step S209, the operator verifies whether a clear tomographic image has been captured without any artifacts. If the tomographic image is clear then the process terminates, if the tomographic image is not clear then the process goes to step S210 to reconstruct the image in accordance with the retrospective ECG method.

[Helical Pitch in the Helical Scan]

Now referring to FIG. 3, there is shown the projection data range when performing a helical scan in case of the apparatus having a plurality of data acquisition systems 105. In the figure an image reconstruction is depicted by extracting the projection data range required for 180 degrees reconstruction (180 degrees plus fan angle=approximately 240 degrees).

The axis of ordinates indicates the direction of body axis at the time of scanning. The axis of abscissa indicates the scanning time starting from the scan start as the revolving angle (π). The helical pitch is indicated as the angle shown as B, and the helical pitch is larger if the angle is more acute. The number of the data acquisition systems 105 is four in case of FIG. 3, those are indicated as DAS1, DAS2, DAS3, and DAS4. The ECG information R is also indicated the parallelogram G encompassed by a dash line indicates the projection data range to be extracted in synchronization with the ECG information R, and the parallelogram G includes four sets of projection data acquired from the data acquisition system 105 of DAS1 to DAS4.

The rectangular frame indicates the reconstructible range. The retrospective ECG imaging method is allowed in the slice range from the projection data extracted from the heartbeat. RECON1 designates the reconstructible range of the first heart beat, RECON2 designates the reconstructible range of the second heart beat, and RECON3 designates the reconstructible range of the third heart beat. If the helical pitch is larger, RECON1 and RECON2 will not overlap in the direction of body axis, to develop a gap in the reconstruction area, so that the ECG synchronization reconstruction is impossible in this section. Accordingly the setting of helical pitch in step S207 of FIG. 2 is important. A reconstruction image can be generated in a given phase of the heart beat by changing or shifting the extraction position of the projection data.

[Image Reconstruction in the Retrospective ECG]

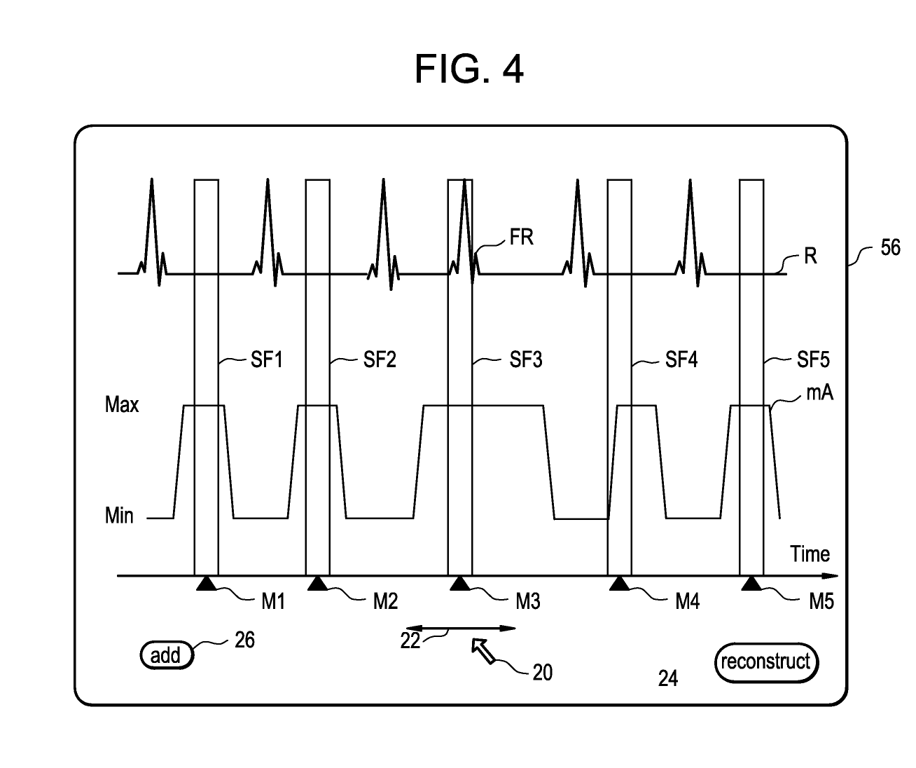

The retrospective ECG in accordance with the present invention as have been described above in step S210 of FIG. 2 will be described in greater details with reference to FIG. 4.

FIG. 4 shows a graphic display screen displayed on the monitor 56 for setting the retrospective ECG configuration. In the graphic display, the abscissa is the time axis, the ECG information R from the electrocardiogram 150 is displayed at the top of the monitor 56 screen, and the tube current mA for changing the radiation intensity of X-ray from the X-ray tube 102 is displayed next thereto. The tube current mA is varying within the range between the MIN value and the MAX value as have been described above in step S204 of FIG. 2. A scan file SF (each scan file is labeled as SF1, . . . , SF5 in the figure) is overlaid on the ECG information R and the tube current mA.

The scan file SF indicates the area of the reconstruction data for forming a tomographic image of the subject, in case in which the number of the data acquisition system 105 is one. As have been described above in the process steps of the prospective ECG method with reference to FIG. 2, a time resolution of 0.2 second to 0.5 second can be achieved for example by combining the gantry 100 revolving at a predetermined constant revolving speed, with a half scan reconstruction for image reconstruction from the projection data of 180 degrees plus fan angle. Each of scan files SF1 to SF5 shown in FIG. 4 has the time width of about 0.3 second, for example.

There are provided button marks M at the bottom of each of scan files SF1 to SF5 (in the figure button marks are indicated as M1 to M5). At the right hand bottom corner of the screen of the monitor 56 there is provided a reconstruction button 24 for switching the screen from the retrospective ECG configuration screen to the tomographic image screen based on the scan file SF having been set.

The operation on the graphic display screen for the retrospective ECG configuration, displayed on the monitor 56 will be described in greater details. It is to be noted here that the following description is merely an exemplary embodiment and that any other forms may be substituted.

The mouse 58 is used to point with the pointer 20 any one of the button mark M1 to button mark M5, on the monitor 56. Then the mouse 58 is clicked to move the button mark M in a transverse direction indicated by an arrow 22 (the arrow is not need to be displayed on the monitor 56, the arrow is indicated in FIG. 4 for merely the explanation) to manipulate the direction of time axis (phase within the RR interval) of any one of the scan files SF1 to SF5. For instance, the operator recognizes that the time zone that the tube current mA of the X-ray tube 102 shifts from MIN to MAX is overlapped with the scan file SF4. In the scan file SF4 the emission intensity of X-ray varies because the tube current mA varies, so that the resultant image may be likely to be unclear as a tomographic image. The operator then points the button mark M4 with the pointer 20 and clicks the mouse 58 to move the button mark M4 to the left hand side of the screen. In this manner the scan file SF4 can be moved to a position where the tube current mA is set to MAX.

The operator may also select a given plurality of button marks M from within the button marks M1 to M5 with the pointer 20 by clicking while holding the shift key down on the keyboard 57 in order to move these scan files SF in the direction of time axis at once. For example, in step S204 of FIG. 2, this technique is effective in case when the tomographic image reconstructed by the prospective ECG method by setting the relative position of the RR interval to 75 percent by the operator is not the expected phase of the heart, and if a tomographic image of another phase, more specifically 73 percent of the relative position is desired.

A specific scan file SF can be selectively deleted. For example, assuming that the operator recognizes that on observation of the ECG information R there are signs of arrhythmia FR, and that the scan file SF3 overlaps on the QRS wave of the arrhythmic heart beat (that is, the relative position of the RR interval is at 0 percent). In such a case the operator selects the button mark M3 with the pointer 20 then press the delete button on the keyboard 57 to erase the scan file SF3 because the tomographic image around the arrhythmia FR is useless. In this case the scan file SF3 disappears from the screen of the monitor 56. On the other hand the button mark M3 still appears on the screen so that the operator may recognize that the scan file SF3 has been deleted. When the scan file SF3 is disappeared from the monitor 56, if the operator double-clicks on the button mark M3, then the scan file SF3 will reappear on the monitor 56, ready to use as the reconstruction data area.

Furthermore, a specific scan file SF can be added. As an example, the operator in step S204 is assumed to set the relative position of the RR interval at the 75 percent. Other than that tomographic image reconstructed, another tomographic image of another phase (for example, the relative position at 71 percent) may be obtained. Then when the operator double-clicks on the add button 26 with the pointer 20, another new scan file SF will appear on the monitor 56. The operator then is allowed to move by the pointer 20 the button mark of this new scan file SF to a phase for example of the relative position at 71 percent. This setting will be used for the reconstruction of another tomographic image as the reconstruction data area.

After manipulating the scan file SF on the monitor screen of the retrospective ECG setting configuration, by the operator clicking on the reconstruction button 24 with the pointer 20, a tomographic image after the scan file SF operation will be displayed. If this tomographic image is not yet the one expected, the screen can be switched to that shown in FIG. 4 by clicking with the pointer 20 on a button that appears when the tomographic image is displayed for the retrospective ECG setting.

In the above description, the button mark M is described displayed on the monitor 56. Alternatively, a specific scan file SF may be deleted or added or moved by clicking directly on a scan file SF, without displaying the button mark M. In addition, a graph of the ECG information R is described displayed on the top of the screen of the monitor 56 and a graph of the tube current mA just beneath. However the order or layout is not limited thereto. Although a scan file SF is displayed overlapped on the ECG information R and the tube current mA, the scan file can be displayed, without being overlapped, apposed along with the graph of the ECG information R.

Furthermore, FIG. 4 has been described as a screen for solely the retrospective ECG setting configuration. However a screen for the retrospective ECG setting configuration can be added on the monitor 56 so as to display a tomographic image at the same time. Even though the screen for the retrospective ECG setting may become smaller, a tomographic image after changing the setting can be verified without need of switching the screen each time a scan file SF is reconfigured.

The present invention as have been described above may be achieved by the operation on the operation console 50 of the X-ray CT apparatus 1, however it is equally possible to execute the process on an independent terminal (a workstation, a personal computer, etc.) which is different from the operation console 50. It will be appreciated by those skilled in the art that various modifications and changes may be equally made on the present invention without departing from the technical spirit and scope thereof.

1. A radiation tomographic imaging apparatus for forming a tomographic image of a subject using the radiation from a source of radiation, comprising:
an electrocardiogram for measuring the heartbeat of the heart of said subject and for outputting as ECG wave signal;
a variable output unit for varying the output of said radiation based on said ECG wave signal; and
a display unit for displaying simultaneously said ECG wave signal, the output of said radiation, and the reconstruction data area for forming said tomographic image.

2. A radiation tomographic imaging apparatus according to claim 1, wherein said display unit displays graphically said ECG wave signal, said output state of the radiation and the reconstruction data area.

3. A radiation tomographic imaging apparatus according to claim 1, further comprising a changing unit for changing the area of said reconstruction data.

4. A radiation tomographic imaging apparatus according to claim 3, wherein said changing unit manipulates the area of reconstruction data displayed on said display unit in the direction of time axis to change said area of reconstruction data.

5. A radiation tomographic imaging apparatus according to claim 4, wherein there is provided a plurality of said areas of reconstruction data, and aid changing means can change a given number of areas in the direction of time axis at the same time.

6. A radiation tomographic imaging apparatus according to claim 3, wherein there is provided a plurality of said areas of reconstruction data, and said changing means deletes or adds at least one of said areas of reconstruction data in order to change said areas of reconstruction data.

7. A radiation tomographic imaging apparatus according to claim 1, further comprising an input unit for inputting a predetermined phase of the heartbeat of said heart, wherein the output of said radiation from said variable output unit varies at the predetermined phase from said ECG wave signal.

8. A radiation tomographic imaging apparatus according to claim 1, wherein said radiation includes X-ray.

9. A radiation tomographic imaging method for forming a tomographic image of a subject by means of the radiation from a source of radiation, comprising:
an ECG wave outputting step for measuring the heartbeat of the heart of a subject to output as ECG wave signal;
a variable outputting step for varying the output of said radiation based on said ECG wave signal;
a determining step for determining whether the tomographic image reconstructed based on the projection data obtained from the varied output of said radiation is good or no good; and
a displaying step for displaying, when the tomographic image is determined to be no good, said ECG wave signal, said output of radiation, and the area of reconstruction data for forming said tomographic image at the same time.

10. A radiation tomographic imaging method according to claim 9, wherein said displaying step graphically displays said ECG wave signal, said output state of radiation, and the area of reconstruction data.

11. A radiation tomographic imaging method according to claim 9, further comprising:
a changing step for changing the areas of reconstruction data.

12. A radiation tomographic imaging method according to claim 11, wherein said changing step manipulates the areas of reconstruction data displayed on said display unit in the direction of time axis to change said areas of reconstruction data.

13. A radiation tomographic imaging method according to claim 12, wherein there is provided a plurality of said areas of reconstruction data, and said changing means can change a given number of the areas in the direction of time axis at the same time.

14. A radiation tomographic imaging method according to claim 11, wherein there is provided a plurality of said areas of reconstruction data, and said changing means deletes or adds at least one of said areas of reconstruction data to change the areas of reconstruction data.

15. A radiation tomographic imaging method according to claim 11, further comprising a tomographic image reconstructing step for reconstructing a tomographic image by using said reconstruction data of the area changed in said changing step, among the projection data obtained by the varied output of said radiation.

16. A radiation tomographic imaging method according to claim 9, further comprising a phase inputting step for inputting a predetermined phase of the heartbeat of said heart,
wherein the output of said radiation is varied at the predetermined phase from said ECG wave signal.

17. A radiation tomographic imaging method according to claim 9, wherein said source of radiation and the moving table for mounting thereon said subject are moved in synchronization, based on said ECG wave signal.

18. A radiation tomographic imaging method according to claim 9, wherein said radiation includes X-ray.
